(19) United States
(12) Patent Application Publication
(10) Pub. No.: US 2009/0233989 A1
(43) Pub. Date: Sep. 17, 2009

(54) Oligonucleotides stimulatory of the mesenchymal stem cell proliferation and uses thereof

(76) Inventor: Ricardo Augustin Lopez, Buenos Aires (AR)

Correspondence Address: DEFILLO & ASSOCIATES, INC., P.O. Box 14104, Clearwater, FL, 33766, US

(21) Appl. No.: 11/921,384

(22) PCT Filed: May 31, 2006
(86) PCT No.: PCT/EP2006/062773

§ 371 Date: Nov. 30, 2007

(30) Foreign Application Priority Data
Jun. 3, 2005 (EP) 05104854.4

(51) Int. Cl. A61K 31/7088

(52) U.S. Cl. 514/44 R

(57) ABSTRACT

Oligonucleotides having the ability to greatly stimulate the proliferation of pluripotent mesenchymal stem cells “in vitro” and “in vivo” of animals, including humans, are disclosed. These oligonucleotides can be used in a wide range of clinical procedures such as (1) regeneration of mesenchymal tissues which have been damaged through acute injury, abnormal genetic expression or acquired disease by inoculation of the ODNs of this invention; (2) treatment of a host with damaged mesenchymal tissue by removal of small aliquots of bone marrow, isolation of their mesenchymal stem cells and treatment of the damaged tissue with MSCs culture-expanded by incubation with one or more of the ODNs of this invention combined with a biocompatible carrier suitable for delivering the MSCs to the damaged body site(s); (3) production “in vitro” of various mesenchymal tissues by directed differentiation of the MSCs culture-expanded by incubation with one or more of the ODNs of this invention, to replace and restore tissue damage or defects with say “in vitro” obtained mesenchymal tissues combined with a biocompatible carrier suitable for delivering the “in vitro” produced tissues to the damaged body site(s); and (4) treatment of a host with abnormal genetic expression with MSCs culture-expanded by incubation with one or more of the ODNs of this invention and transformed by genetic engineering procedures to express a protein able to replace the genetic deffect.

### CROSS REFERENCE TO RELATED APPLICATION

This application is a national stage of PCT/EP02006/0062773 filed May 31, 2006 and based upon EPO Patent Application No. 05104854.4 filed Jun. 3, 2005, under the International Convention.

### FIELD OF THE INVENTION

This invention relates to the use of oligonucleotides for the manufacture of pharmaceutical compositions which induce “in vitro” and “in vivo” proliferation of MSCs. More in details it refers to oligonucleotides having about 14 to 100 nucleotides that have the ability to greatly stimulate the proliferation of MCSs in animals, including human.

### BACKGROUND OF THE INVENTION

These oligonucleotides have the ability to greatly stimulate the proliferation of pluripotent mesenchymal stem cell “in vitro” and “in vivo” of animals, including humans are disclosed. They can be used in a wide range of clinical procedures such as (1) regeneration of mesenchymal tissues which have been damaged through acute injury, abnormal genetic expression or acquired disease by inoculation of the ODNs of this invention; (2) treatment of a host with damaged mesenchymal tissue by removal of small aliquots of bone marrow, isolation of their mesenchymal stem cells and treatment of the damaged tissue with MSCs culture-expanded by incubation with one or more of the ODNs of this invention combined with a biocompatible carrier suitable for delivering the MSCs to the damaged body site(s); (3) production “in vitro” of various mesenchymal tissues by directed differentiation of the MSCs culture-expanded by incubation with one or more of the ODNs of this invention, to replace and restore tissue damage or defects with say “in vitro” obtained mesenchymal tissues combined with a biocompatible carrier suitable for delivering the “in vitro” produced tissues to the damaged body site(s); and (4) treatment of a host with abnormal genetic expression with MSCs culture-expanded by incubation with one or more of the ODNs of this invention and transformed by genetic engineering procedures to express a protein able to replace the genetic deffect.

Methods for generation of mesenchymal tissues in an area of an animal where say tissues are deficient are provided. They consist in the local or systemic administration of a composition comprising one or more of the oligonucleotides to the animal, in a pharmaceutically acceptable carrier. The composition is administered in an amount effective to induce repair at the damaged site.

Methods for extensive proliferation of MSCs of an animal “in vitro” are also provided. They consist in incubation of small aliquot of bone marrow extracted from the animal, “in vitro” incubation of the cells present in say aliquot of bone marrow in a culture media containing an effective concentration of one or more of the ODNs of this invention for a time period sufficient to obtain the amount of cells necessary for a successful medical treatment.

Mesenchymal stem cells (MSC) also known as marrow stromal cells or mesenchymal progenitor cells, are blast cells found, among other body sites, in bone marrow, blood, dermis and periosteum that are capable of differentiating “in vitro” into bone, cartilage, fat, tendon, muscle, hepatic, renal, cardiac, and neural cells depending upon influences from various bioactive factors (Alhadlaq, A and Mao, J. J. Stem Cells Dev. 13:436, 2004). Also, cultured MSC have the ability to differentiate into different cell specific lineages when placed “in vivo” at the site of a given damaged tissue. Therefore, MSC are a valuable material for medical applications such as systemic transplantation for systemic diseases, local implantation for local tissue defects, gene therapy protocols or generation of transplantable tissues and organs in tissue engineering protocols. In order to isolate MSC, it is necessary to separate them from other cells in the bone marrow or other MSC source. Bone marrow cells may be obtained from iliac crest, femora, tibiae, spine, rib or other medullar spaces. Other sources of MSC include embryonic yolk sac, placenta, umbilical cord, fetal and adolescent skin, blood and other mesenchymal stem cell tissues. A number of processes have been developed for isolation, purification and replication of MSC in cultures (Alhadlaq, A and Mao, J. J. Stem Cells Dev. 13:436, 2004). However, limited proliferative potential of MSC in addition to a very low number in the bone marrow or other sources have so far limited their clinical applications. Immortalization of MSC using viral transforming proteins has been described. However, transformed cells are unlikely to be accepted for standard medical treatments own to its possible tumor inducing potential.

### SUMMARY OF THE INVENTION

We now disclose that oligonucleotides having about 14 to 100 nucleotides, preferably from 14 to 40, even more preferably from 14 to 26, are compounds with potent activity to stimulate the proliferation of pluripotent mesenchymal stem cell of animals, including humans, “in vitro” and “in vivo”. According to preferred embodiments, the oligonucleotides to be used in accordance to the present invention consists of 25 or less nucleotides and are not antisense.

According to one embodiment of the invention, a preferred oligonucleotide has at least one subsequence with the following composition: PyNTTTTNT, wherein Py is C or T and wherein N is any deoxyribonucleotide.

Suitable oligonucleotides may be selected from the group consisting of:

| | |
|---|---|
| TCATCATTTTGTCATTTTGTCATT; | (SEQ ID N^{o} 2) |
| TCCTCCTTTTGTCCTTTTGTCCTT; | (SEQ ID N^{o} 3) |
| TCTTCTTTTTGTCTTTTTGTCTTT; | (SEQ ID N^{o} 4) |
| TTGTTGTTTTGTTGTTTTGTTGTT; | (SEQ ID N^{o} 7) |
| TCATTTTTTTGTTTTTTTGTCATT; | (SEQ ID N^{o} 10) |
| TCATTGTTTTGTTGTTTTGTCATT; | (SEQ ID N^{o} 11) |
| TCATTCTTTTGTTCTTTTGTCATT; | (SEQ ID N^{o} 12) |
| TCATTATTTTGTTATTTTGTCATT; | (SEQ ID N^{o} 15) |
| TCATCCTTTTGTCCTTTTGTCATT; | (SEQ ID N^{o} 17) |
| TCATCTTTTTGTCTTTTTGTCATT; | (SEQ ID N^{o} 18) |
| CATTTTGTTTTTTTTTTTTTTTTT; | (SEQ ID N^{o} 20) |
| TTCATTTTGTTTTTTTTTTTTTTT; | (SEQ ID N^{o} 21) |
| TTTTCATTTTGTTTTTTTTTTTTT; | (SEQ ID N^{o} 22) |
| TTTTTTCATTTTGTTTTTTTTTTT; | (SEQ ID N^{o} 23) |
| TTTTTTTTCATTTTGTTTTTTTTT; | (SEQ ID N^{o} 24) |
| TTTTTTTTTTCATTTTGTTTTTTT; | (SEQ ID N^{o} 25) |
| TTTTTTTTTTTTCATTTTGTTTTT; | (SEQ ID N^{o} 26) |
| TTTTTTTTTTTTTTCATTTTGTTT; | (SEQ ID N^{o} 27) |
| TTTTTTTTTTTTTTTTCATTTTGT; | (SEQ ID N^{o} 28) |
| TTTTCATTTTGTCATTTTGTTTTT; | (SEQ ID N^{o} 29) |
| TCATCAATTTGTCAATTTGTCATT; | (SEQ ID N^{o} 30) |
| ACATCATTTTGTCATTTTGTCATT; | (SEQ ID N^{o} 46) |
| CCATCATTTTGTCATTTTGTCATT; | (SEQ ID N^{o} 47) |
| GCATCATTTTGTCATTTTGTCATT; | (SEQ ID N^{o} 48) |
| TAATCATTTTGTCATTTTGTCATT; | (SEQ ID N^{o} 49) |
| TTATCATTTTGTCATTTTGTCATT; | (SEQ ID N^{o} 50) |
| TGATCATTTTGTCATTTTGTCATT; | (SEQ ID N^{o} 51) |
| TCCTCATTTTGTCATTTTGTCATT; | (SEQ ID N^{o} 52) |
| TCTTCATTTTGTCATTTTGTCATT; | (SEQ ID N^{o} 53) |
| TCAACATTTTGTCATTTTGTCATT; | (SEQ ID N^{o} 55) |
| TCACCATTTTGTCATTTTGTCATT; | (SEQ ID N^{o} 56) |
| TCAGCATTTTGTCATTTTGTCATT; | (SEQ ID N^{o} 57) |
| TCATCATTTTGTCATTTTGTAATT; | (SEQ ID N^{o} 58) |
| TCATCATTTTGTCATTTTGTTATT; | (SEQ ID N^{o} 59) |
| TCATCATTTTGTCATTTTGTGATT; | (SEQ ID N^{o} 60) |
| TCATCATTTTGTCATTTTGTCCTT; | (SEQ ID N^{o} 61) |
| TCATCATTTTGTCATTTTGTCTTT; | (SEQ ID N^{o} 62) |
| TCATCATTTTGTCATTTTGTCAAT; | (SEQ ID N^{o} 64) |
| TCATCATTTTGTCATTTTGTCACT; | (SEQ ID N^{o} 65) |
| TCATCATTTTGTCATTTTGTCAGT; | (SEQ ID N^{o} 66) |
| TCATCATTTTGTCATTTTGTCATA; | (SEQ ID N^{o} 67) |
| TCATCATTTTGTCATTTTGTCATC; | (SEQ ID N^{o} 68) |
| TCATCATTTTGTCATTTTGTCATG; | (SEQ ID N^{o} 69) |
| TCATCAATTGGTCAATTGGTCATT; | (SEQ ID N^{o} 75) |
| TCATCAACTGGTCAACTGGTCATT; | (SEQ ID N^{o} 77) |
| TCATCATTGTGTCATTGTGTCATT; | (SEQ ID N^{o} 84) |
| TCATCATTTGGTCATTTGGTCATT; | (SEQ ID N^{o} 87) |
| TGCTGCTTTTGTGCTTTTGTGCTT; | (SEQ ID N^{o} 91) |
| TCATCATCTTGTCATCTTGTCATT; | (SEQ ID N^{o} 95) |
| TCATCATGTTGTCATGTTGTCATT; | (SEQ ID N^{o} 96) |
| GGGGGTCTTTTTTTCTTTTTTTTT; | (SEQ ID N^{o} 107) |
| TTTTTTCTTTTTTTCTTTTTTGGG; | (SEQ ID N^{o} 108) |
| AAAAATCTTTTTTTCTTTTTTTTT; | (SEQ ID N^{o} 109) |
| TGCTGCTTTTATGCTTTTATGCTT; | (SEQ ID N^{o} 110) |
| TCATCATTCTGTCATTCTGTCATT; | (SEQ ID N^{o} 111) |
| TTTTTTCTTTTTTTCTTTTTTTTT; | (SEQ ID N^{o} 112) |
| TGCTGCTTTTCTGCTTTTCTGCTT; | (SEQ ID N^{o} 113) |
| TTTTTTCTTTTCTTTTTTTTTTTT; | (SEQ ID N^{o} 114) |
| TTTTTCCTTTTTTCCTTTTTTTTT; | (SEQ ID N^{o} 115) |
| CCCCCTCTTTTTTTCTTTTTTTTT; | (SEQ ID N^{o} 116) |
| TTTTTTCTTTTTCTTTTTTTTTTT; | (SEQ ID N^{o} 117) |
| TTTTTTCTTTTTTTCTTTTTTCCC; | (SEQ ID N^{o} 118) |
| TTTTTTCTTTTTCTCTTTTTCTCT; | (SEQ ID N^{o} 119) |
| TTTTTGCTTTTTTGCTTTTTTTTT; | (SEQ ID N^{o} 120) |
| TTTTTACTTTTTTACTTTTTTTTT; | (SEQ ID N^{o} 121) |
| TCATAATTTTGTAATTTTGTCATT; | (SEQ ID N^{o} 122) |
| TTTTTTCTTTTTTTCTTTTTTAAA; | (SEQ ID N^{o} 123) |
| TTTTTTCTTTTTTCTTTTTTTTTT; | (SEQ ID N^{o} 124) |
| TTTTTTTTTTTTCATTTTGTGGGG; | (SEQ ID N^{o} 125) |
| TTTTTTTTTTTTCATTTTGTTTTG; | (SEQ ID N^{o} 126) |
| GGGTTTTTTTTTCATTTTGTTTTT; | (SEQ ID N^{o} 127) |
| GTTTTTTTTTTTCATTTTGTTTTG; | (SEQ ID N^{o} 128) |
| TTTTTTTTTTTTCATTTTGTTTGG; | (SEQ ID N^{o} 129) |
| TTTTTTTTTTTTCATTTTGTTTTA. | (SEQ ID N^{o} 130) |
| TTTTTTTTTTTTCATTTTGTTTGA; | (SEQ ID N^{o} 131) |
| TTTTTTTTTTTTCATTTTGTTTAG; | (SEQ ID N^{o} 132) |
| TTTTTTTTTTTTCATTTTGTTTAA. | (SEQ ID N^{o} 133) |

It is an object of the present invention to provide one or more of the oligonucleotides of this invention to an animal with a tissue deficiency to produce normal new normal tissue where it is needed.

Another object of the present invention is to provide a method to enhance the replication of pluripotent mesenchymal stem cell “in vitro” so that an adequate number of these cells can be obtained for successful medical treatments.

These objects will become apparent to those skilled in the art.

The first of the above objects is achieved by providing a method for tissue regeneration at a site of an animal where the tissue is deficient and which consists in the administration of an effective amount of a composition comprising one or more of the oligonucleotides of this invention to the animal, locally at the site or systemically as needed in each case, in a pharmaceutically acceptable carrier, the composition being administered in an amount effective to induce tissue regeneration at the site.

This aspect of the invention enables the generation of normal tissue in general or locally as required. Pre-clinical results using as example some of the osteogenic ODNs of this invention described below show tissue formation in tissue defects in rats.

The second of the above objects is achieved by providing a method to enhance the replication of pluripotent mesenchymal stem cell “in vitro” which consists in obtention of bone marrow cells from a suitable source like iliac crest, femora, tibiae, spine, rib or other medullar spaces, culturing these cells “in vitro” using a culture medium to the art supplemented with one or more of the oligonucleotides of this invention for a time period sufficient to obtain the amount of cells necessary for a successful medical treatment.

In particular, the oligonucleotides according to the present invention can be used for the manufacture of a medicament for the treatment of the tissue damage own to a heart stroke, for the treatment of skeletal deficiency or alteration, for the treatment of the tissue damage own to a cartilage deficiency or alteration, for the treatment of the tissue damage own to a nervous system deficiency or alteration, for the treatment of the tissue damage own to a adipose tissue deficiency or alteration, for the treatment of the tissue damage own to a liver deficiency or alteration, for the treatment of the tissue damage own to a muscle deficiency or alteration, for the treatment of the tissue damage own to a skin deficiency or alteration, for the treatment of the tissue damage own to a burn accident, for the treatment of the tissue damage own to a pancreas deficiency or alteration, for the treatment of the tissue damage own to a mucosal deficiency or alteration, for the treatment of the tissue damage own to a blood vessel deficiency or alteration, for the treatment of the tissue damage own to a blood deficiency or alteration and/or for improvement of bone marrow engraftment.

The oligonucleotides for use in the present invention may have any kind of modification of the natural (phosphodiester) phosphate backbone, as for instance a phosphate backbone modification on the 5′ inter-nucleotide linkages or a phosphate backbone modification on the 3′ inter-nucleotide linkages, or they may have at least one of the internucleotide linkages as a phosphorotioate linkage.

The medicament containing at least one of the above discussed oliconucleotides is preferably administered to a human, the oligonucleotide being preferably present in amounts of 1 μg to 100 mg per dose. The medicament may be administered by intradermic, intramuscular or intravenous injection or by oral, intranasal, anal, vaginal, or trans-dermal route; the medicament may be selected from the group consisting of liquid, gel and lyophilized formulations. The oligonucleotide may be included in a pharmaceutically acceptable carrier and/or may be encapsulated in a slow release delivery vehicle.

The oligonucleotide may also be administered to a subject combined with a device aimed to aid in the treatment of a tissue or organ deficiency or alteration.

According to an embodiment, the mesenchymal stem cells are obtained from bone marrow or from periosteum. According to a further embodiment, the mesenchymal stem cells differentiate into cells of more than one tissue cell type, said tissue being preferably selected from bone, cartilage, adipose, tendon, ligament, dermis, epidermis, neuronal, cardiac, renal, glandular and hepatic tissue.

### BRIEF DESCRIPTION OF THE DRAWINGS

FIG. 1a shows the replication of MSCs extracted from the femoral bone marrow of rats stimulated or not in the presence of ODN 504 (SEQ ID N^{o}2).

FIG. 1b shows the MSC morphology of cells in controls and ODN IMT504 treated cultures.

FIG. 2a shows adipocytes, fibroblasts and macrophages in LTBM culture supplemented with ODN IMT504.

FIG. 2b shows calcium deposits in osteoblasts obtained from rat bone marrow cells in osteogenic medium supplemented with ODN IMT504.

FIG. 3 shows the MSCs replication capacity in cultures supplemented with CpG ODNs prototypes ODN 2006 and ODN 2216 as compared with the MSCs replication capacity in a culture supplemented with ODN 504.

FIG. 4 shows MSCs colony forming units obtained by incubation of bone marrow cells of rats inoculated or not with ODN 504.

FIG. 5a shows the radiographic evolution of a defect experimentally provoked in the tibial bone of a rat injected with the ODN IMT504 as compared with a rat injected with placebo after 4 weeks of treatment.

FIG. 5b shows the histological analysis of a defect experimentally provoked in the tibial bone of a rat injected with the ODN IMT504 as compared with a rat injected with placebo after 4 weeks of treatment.

### DESCRIPTION OF THE PREFERRED EMBODIMENTS

Repairing a tissue means promoting the formation of morphologically normal, mature tissue only at a site where there is a tissue deficiency that needs to be replaced.

By “animal” is meant any animal having a vertebrate structure, preferably a mammal, and most preferably a human.

A “subject” refers to an animal of the order primate, including humans.

As used herein, the term “oligonucleotide” or “oligo” shall mean multiple nucleotides (i.e. molecules comprising a sugar, e.g. ribose or deoxyribose, linked to a phosphate group and to an exchangeable organic base, which is either a substituted pyrimidine (e.g. cytosine (C), thymine (T) or uracil (U)) or a substituted purine (e.g. adenine (A) or guanine (G)).

The term “oligonucleotide” as used herein refers to both oligoribonucleotides (ORNs) and oligodeoxyribonucleotides (ODNs). The term “oligonucleotide” shall also include oligonucleosides (i.e. an oligonucleotide minus the phosphate) and any other organic base containing polymer. Oligonucleotides can be obtained from existing nucleic acid sources (e.g. genomic or cDNA), but are preferably synthetic (e.g. produced by oligonucleotide synthesis).

An “oligonucleotide” refers to multiple nucleotides linked by phosphodiester bonds.

An “immunostimulatory oligonucleotide” refers to an oligonucleotide which stimulates (i.e. has a mitogenic effect on, induces, increases or decreases cytokine expression by) a cell of the immune system (i.e. a lymphocyte or a macrophage) in a statistically significant manner.

A “CpG” refers to a cytosine-guanine dinucleotide.

A “CpG oligonucleotide” refers to an oligonucleotide which stimulates a cell of the immune system, and whose immunostimulatory activity critically depends on the presence of at least one CpG in its sequence.

A “non-CpG oligonucleotide” refers to an oligonucleotide that stimulates a cell of the immune system, and whose immunostimulatory activity does not critically depend on the presence of a CpG in its sequence.

### MODES FOR CARRYING OUT THE INVENTION

### Example 1

### Materials and Methods

The following materials and methods were generally used throughout the examples.

### Oligonucleotides

Oligonucleotides having phosphorothioate internucleotide linkages were purchased, purified by high-pressure liquid chromatography (HPLC), from Operon Technologies (Alameda, Calif.) or Annovis (Aston, Pa.) or Oligos Etc (Bethel, Me.). ODNs were suspended in depyrogenated water, assayed for LPS contamination using the Limulus test and kept at −20° C. until used. Purity was assessed by HPLC and PAGE assays. ODN preparations were used if LPS levels were undetectable.

### 2) Animal Experiments

2a—Bone Marrow (BM) Extraction: Rat BM-derived MSCs were harvested from 8 to 12 weeks old (about 350 g) male Spragüe Dawley rats. Animals were anesthetized by intraperitoneal (i. p.) injection of a mixture of ketamine (50 mg/kg) and xilacine (5 mg/kg). After removing epiphyses and gaining access to the marrow cavities, whole BM plugs were flushed out from femoral bones using a 1 ml syringe with α-MEM medium supplemented with 100 IU/ml gentamicin and 25 μg/ml amphotericin.

2b—“In Vivo” MSCs Replication Stimulation by Oligonucleotide Treatment: Young (8-12 weeks old) adult male Spragüe Dawley rats weighing about 350 g were subcutaneously injected with 300 μl of PBS containing 250 μg of the oligonucleotide IMT504 (Seq. ID N^{o}2) (treated animals) or 300 μl of PBS (control animals) once a day for five days. Two days after this, the animals were sacrificed and the BM-derived MSCs extracted as described above.

2c—“In Vivo” Osteogenesis Stimulation by Oligonucleotide Treatment: Young (8-12 weeks old) adult male Spragüe Dawley rats weighing about 350 g were used. Animals were anesthetized by intraperitoneal (i. p.) injection of a mixture of ketamine (50 mg/kg) and xilacine (5 mg/kg). After skin overlying the hind limb was shaved and sterilized. A 1.5 cm longitudinal incision was performed in the tibia forefront zone. To generate a defect in the bone, an osteotomy was made using a low-speed dental drill attached to a round diamond saw under saline irrigation. For the osteotomy, a site free of muscular insertions was selected 15 mm below the ankle. The wound was deep enough to reach the bone marrow. A single dose (as stated in each experiment) of ODN in 3 μl of methylcellulose 1% was introduced into the defect made in the right tibia. As a control, the same volume (3 μl) of vehicle was introduced into the defect made in the left tibia.

Fracture callus formation was evaluated radiographically on days 0, 21 and 28. On day 28, animals were euthanized under ether atmosphere, and the tibias removed and photographed. After this, the tibias were fixed in 10% formol solution, decalcified in 10% EDTA solution, and embedded in paraffin. A longitudinal section of each tibia was cut, stained with the Masson's trichromic technique to visualize calcified zones and examined under a light microscope.

### 3) Tissue Culture

3a—MSCs Colony Forming Units Determination (Castro-Malaspina H., Gay R. E., Resnick G., Kapoor N., Meyers P., Chiarieri D., McKenzie S., Broxmeyer H. E., Moore M. A. Characterization of human bone marrow fibroblast colony-forming cells (CFU-F) and their progeny Blood. 1980; 56(2): 289-301): 2×10⁶ cells extracted from femoral bones as described above were cultured in 25 cm² culture dish containing 10 ml of α-MEM medium supplemented with 100 IU/ml gentamicin, 25 μg/ml amphotericin, 2 mM L-glutamine and 20% fetal calf serum. Incubation was in 5% CO2 at 37° C. After seven days unattached cells were wash out and fresh medium added. ODN treated cultures contained 1 μM of the indicated ODN during the first seven days of culture. Incubation proceeded until day 14. After this, attached cells were washed twice with PBS, fixed with methanol and stained using the May-Grungwald-Giemsa technique. Colonies were observed and counted using an optical microscope.

3b—Long Term Bone Marrow (LTBM) Culture: This culture technique was used in order to assess the differentiation capacity of the cultured MSCs (Gartner S., Kaplan H. S. Long-term culture of human bone marrow cells. Proc Natl Acad Sci U S A. 1980; 77(8): 4756-4759). 10×10⁶ extracted from femoral bones as described above were cultured in 25 cm² culture dish containing 10 ml of α-MEM medium supplemented with 100 IU/ml gentamicin, 25 μg/ml amphotericin, 2 mM L-glutamine, 12.5% fetal calf serum, 12.5% horse serum, and 10⁻⁸ M hydrocortisone. Incubation was in 5% CO2 at 37° C. for 28 days. Unattached cells were eliminated every seven days by centrifugation of the cell supernatant. After this, half of the supernatant was replaced by fresh medium and returned to the culture dish. ODN treated cultures contained 1 μM of the indicated ODN during the first seven days of culture.

After this, the stimulus was progressively diluted with the medium replacement. After incubation, attached cells were washed twice with PBS, fixed with methanol and stained using the May-Grunwald-Giemsa technique. Cells were observed using an optical microscope.

3c—Osteogenic Differentiation of the MSCs “In Vitro” (Bruder S. P, Jaiswal N., Haynesworth S. E. Growth kinetics, self-renewal, and the osteogenic potential of purified human mesenchymal stem cells during extensive subcultivation and following cryopreservation. J Cell Biochem. 1997; vol. 64, p. 278): 2×10⁶ cells extracted from femoral bones as described above were cultured in 25 cm² culture dish containing 10 ml of D-MEM medium supplemented with 100 IU/ml gentamicin, 2.5 μg/ml amphotericin, 2 mM L-glutamine and 10% fetal calf serum. Cultures also contained 1 μM of the indicated ODN. After three days culture unattached cells were wash out and fresh medium and ODN were added. After seven days more of culture, cells were trypsinized and seeded in a new culture dish at 3000 cells/cm² containing the differentiating medium. This medium consisted in D-MEM supplemented with 10−8 dexametazone, 0.2 mM ascorbic acid and 10 mM β-glicerophosphate. The cell supernatant was replaced every three days with fresh medium. After 21 days attached cells were washed twice with PBS, fixed with methanol and stained using the Alizarin Red technique to visualize calcium deposits.

### Example 2

### ODN Stimulation of the MSCs Replication “In Vitro”

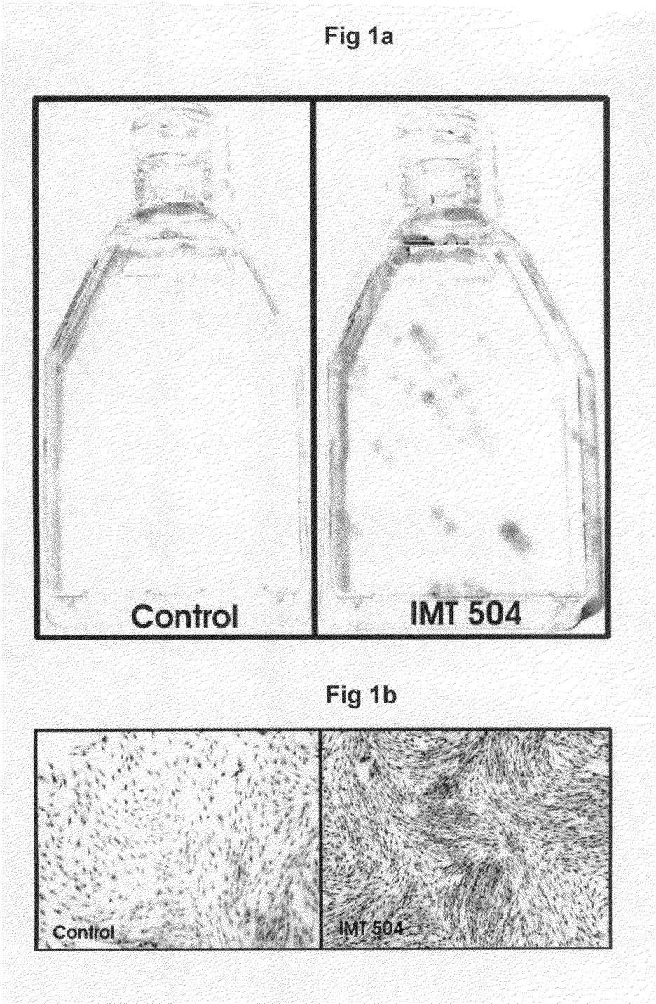

The oligonucleotide used in these experiments was IMT504 (SEQ ID N^{o}2). This oligonucleotide is 24 nucleotides long; its nucleotide sequence is 5′-TCATCATTTTGTCATTTTGTCATT-3′, and all the DNA (natural) phosphodiester bonds have been replaced with phosphorothioate bonds to protect it from enzymatic degradation. FIG. 1a shows that the replication of MSCs extracted from the femoral bone marrow of rats are greatly stimulated in the presence of ODN 504. FIG. 1b shows the typical MSC morphology of cells in controls and treated cultures. Many other oligonucleotides (e.g.: SEQ ID N^{o}3, SEQ ID N^{o}4, SEQ ID N^{o}7 and SEQ ID N^{o}10) were assayed with similar results.

### Example 3

### MSCs Replicated “In Vitro” Under ODN Stimulation Conserve the Multipotent Differentiation Capacity

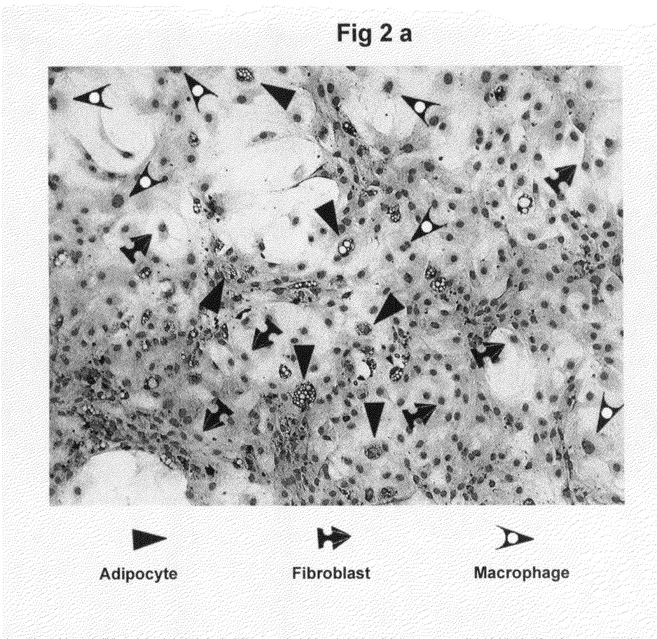

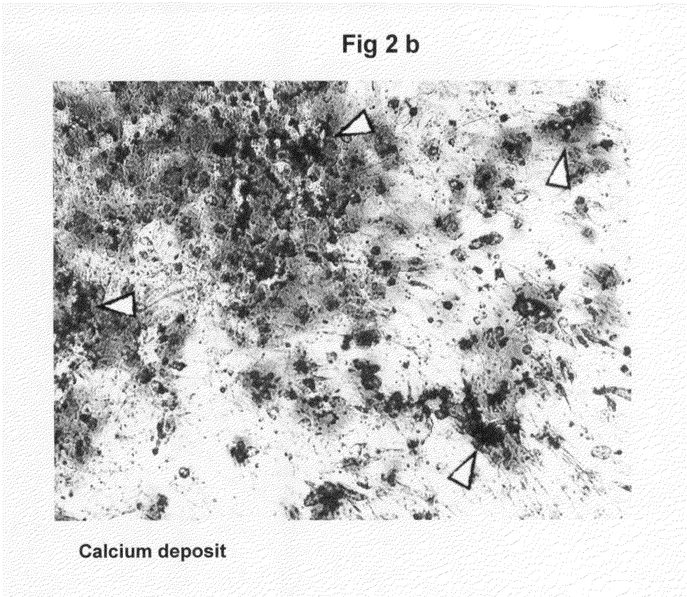

FIG. 2a shows that MSCs replicated “in vitro” under ODN stimulation could differentiate in LTBM culture to adipocytes, fibroblasts and macrophages as expected for normal MSCs (Gartner S., Kaplan H. S. Long-term culture of human bone marrow cells. Proc Natl Acad Sci USA. 1980; 77(8): 4756-4759). On the other hand, FIG. 2b shows that in osteogenic medium (Bruder S. P, Jaiswal N., Haynesworth S. E. Growth kinetics, self-renewal, and the osteogenic potential of purified human mesenchymal stem cells during extensive subcultivation and following cryopreservation. J Cell Biochem. 1997; vol. 64, p. 278) MSCs replicated “in vitro” under ODN stimulation could differentiate into osteoblasts that form calcium deposits.

### Example 4

CpG ODNs are Poor Stimulators of the MSCs Replication “in vitro”

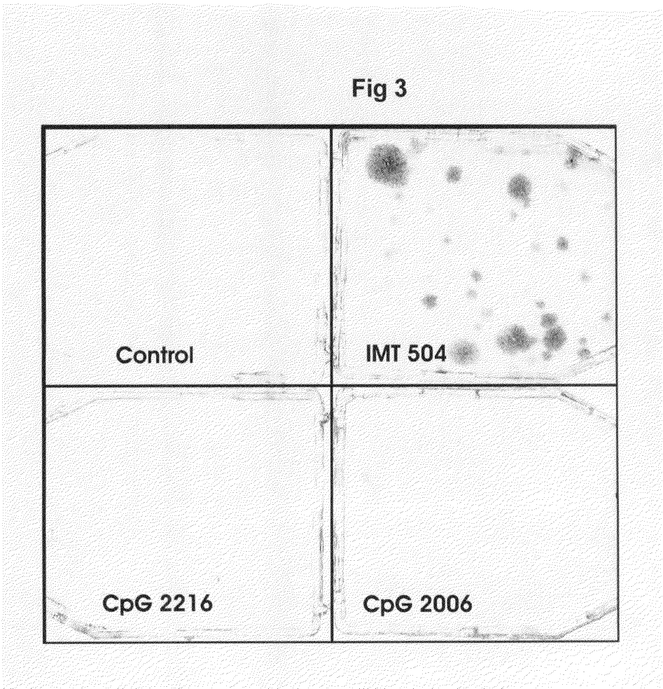

FIG. 3 shows that CpG ODNs prototypes ODN 2006 and ODN 2216 (Krieg, A. M. GpG motifs in bacterial DNA and their immune effects. 2002. Annu. Rev. Immunol. 20, 709-760) are poor stimulators of the MSCs replication as compared with ODN 504. Many other CpG ODNs were assayed with identical results.

### Example 5

ODN Stimulation of the MSCs Replication “in vivo”

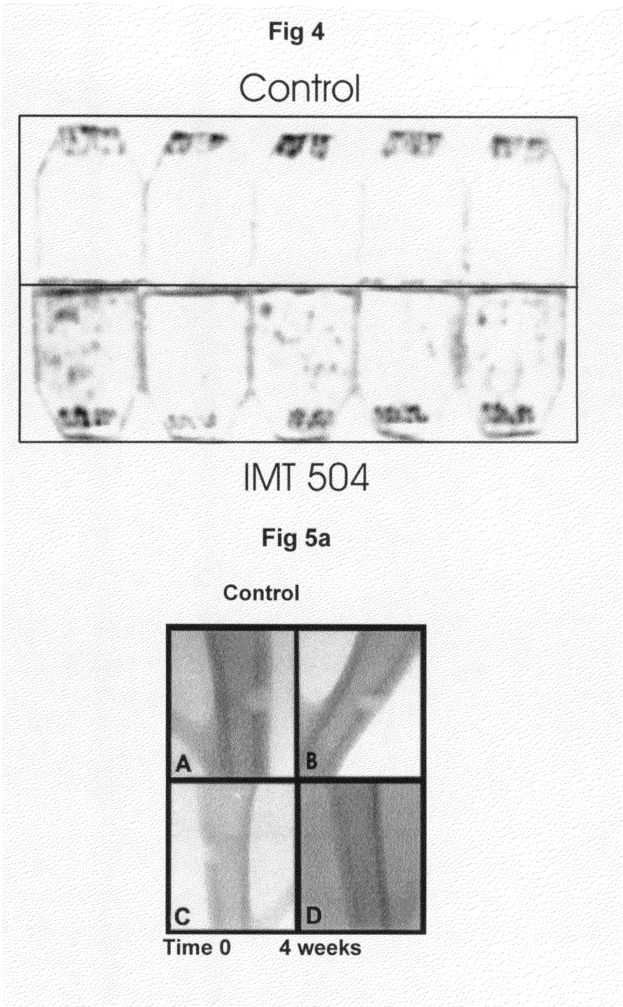

FIG. 4 shows that bone marrow of rats inoculated with ODN 504 have a very high level of MSCs colony forming cells as compared with rats inoculated with placebo. This result indicates that the stimulation of the MSCs replication by ODNs also occurs “in vivo”.

### Example 6

ODN Stimulation of the Osteogenesis “in vivo”

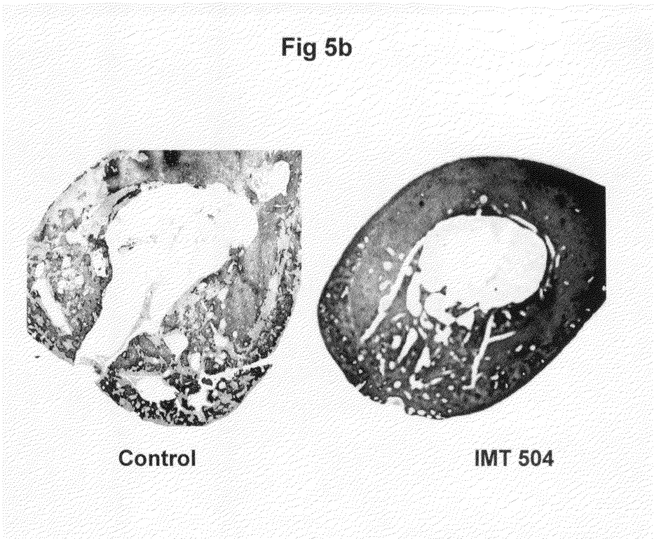

Since stimulation of the replication of MSCs could be induced by the ODN and since MSCs are osteogenic when injected in an animal (Bruder S P, Fink D J, Caplan A I. Mesenchymal stem cells in bone development, bone repair, and skeletal regeneration therapy. J Cell Biochem. 1994 November; 56(3):283-94. Review), the osteogenic potential of the MSCs over-replicated “in vivo” by ODN injection was assayed. FIGS. 5a and 5b shows that an experimental defect provoked in the tibial bone of rats is rapidly repaired in animals treated with the ODN IMT504 as compared with rats injected with placebo.

### Example 7

### A) ODN IMT504 Stimulation of Damage Liver Repair After Sepsis

Since stimulation of the replication of MSCs could be induced by the ODN and since MSCs accelerate hepatic tissue repair when injected in an animal (Sotnikova N V, Stavrova L A, Gur'antseva L A, Khrichkova T Y, Fomina T I, Vetoshkina N V, Dubskaya T Y, Sergeeva S A, Epshtein O I, Ermolaeva L A, Dygai A M, Gol'dberg E D. Mechanisms of the effects of granulocytic CSF on tissue reparation during chronic CCl4-induced damage to the liver. Bull Exp Biol Med. 2005 November; 140(5):644-7; Fang B, Shi M, Liao L, Yang S, Liu Y, Zhao R C. Systemic infusion of FLK1(+) mesenchymal stem cells ameliorate carbon tetrachloride-induced liver fibrosis in mice. Transplantation. 2004 Jul. 15; 78(1):83-8.), the hepatic tissue repair potential of the MSCs over-replicated “in vivo” by ODN injection was assayed using a model of experimental liver damage provoked in rats by sepsis. Fig. . . . shows the presence of liver damage in animals injected with placebo and the absence of liver damage in animals injected with ODN IMT504.

### Material and Methods

2d—Induction of Multiple Organ Failure by Sepsis: Young (6-9 weeks old) adult male Sprague Dawley rats weighing about 350 g were used. Bacteria were Escherichia coli Strain O45K1H10. Bacteria were grown using standard brain hart infusion broth at 37° C., during 4 h with continuous shaking. After this, bacteria were seeded in Muller Hinton agar (1.5%) and incubated at 37° C. over night. Subcultures were performed in Trypticase Soy Agar (1.5%) in order to isolate smooth colonies. Colonies were seeded in TSB broth and 4 ml of the culture were seeded in TSB agar and incubated at 37° C. during 24 h. Colonies were suspended in PBS and washed three times with PBS and suspended in PBS. The bacterial concentration was determined by reading OD600 in a spectrophotometer. The working concentration was 1×10⁹ bacteria/ml. Rats were intraperitoneally inoculated at day 0 with 1 ml of the bacterial suspension. Treated animals were subcutaneously injected with 6 mg in 0.3 ml of PBS, 4 times (total 24 mg), and each injection separated 3 h from the previous one at day 1. Control animals were injected with PBS using the same protocol. At day 4 post-bacterial inoculation animals were sacrificed, livers removed and studied by histo-pathological analysis using standard techniques.

### Example 8

Recovering from Peripheral Nerve Crush by Treatment With ODN IMT504

Since stimulation of the replication of MSCs could be induced by ODNs of the PyNTTTTGT class and since MSCs accelerate recovering in a rat model of peripheral nerve crush (Coronel M F, Musolino P L, Villar M J. Selective migration and engrafment of bone marrow mesenchymal stem cells in rat lumbar dorsal root ganglia after sciatic nerve constriction. Neurosci Lett. 2006; 405:5-9), the recovering after nerve crush induced by IMT504 injection was assayed in the same rat model. In this model of neuropathic pain (sciatic nerve crush), intramuscularly injected IMT504 showed significant protective effects as reveled by two tests (Von Frey and Choi) which measure allodynia: an exaggerated response to otherwise non-noxious stimuli (FIGS. 6A and B).

### Material and Methods

Nerve Injury Model: Experiments were performed on adult (200-300 g) Sprague Dawley rats. Animals were anaesthetized with chloral hydrate (350 mg/kg, i.p.) and their right sciatic nerve was exposed and dissected free from the surrounding tissue at the mid-thigh level. It was then crushed using a jeweller's forcep during a 5 minutes period of time. The extent of crush injury of each nerve was confirmed after dissection under a surgery microscope using a 10 mm ruler, and also by microscopical observation of 16 □m sections stained with neutral red.

Behavioral Assessment: Behavioral testing was performed during daytime (9.00-18.00) in all animals before surgery (day 0) and 1, 3, 7, 14, 21, 28 and 56 days after transplantation and nerve crush. The animals were placed in their acrylic testing chambers for 15 min for adaptation, and mechanical sensitivity was assessed with von Frey hairs (Stoelting, WoodDale, Ill., USA). The hairs were applied in ascending order (1, 2, 4, 6, 8, 10, 15, 23 g) onto the plantar surface of both ipsilateral and contralateral hindpaws (S. Chaplan, F. Bach, J. Pogrel, J. Chung, T. Yaksh, Quantitative assesment of tactile allodynia in the rat paw, J. Neurosci., 16 (1994) 7711-7724.). Each hair was delivered three times with 5 s intervals. The lowest force at which application elicited a paw withdrawal was taken as the mechanical response threshold. A paw withdrawal reflex obtained with 6 g or less was considered as an allodynic response. Cold sensitivity of the hindpaw to acetone (Choi test) (Y. Choi, Y. W. Yoon, H. S. Na, S. H. Kim, J. M. Chung, Behavioral signs of ongoing pain and cold allodynia in a rat model of neuropathic pain, Pain, 59 (1994) 369-376.) was quantified by foot withdrawal frequency. Hundred [symbol] l of acetone were applied to the plantar surface of the paw using a plastic tubule connected to a 1 ml syringe. Acetone was applied five times to each paw at an interval of at least 5 min. The number of brisk foot withdrawals was recorded.

1-22. (canceled)

23. A method for inducing repair of tissue damage to a patient, the method comprising:
applying to the patient a medicament comprising a non-antisense oligonucleotide having about 14 to 40 nucleotides and having at least one subsequence of formula PyNTTTTNT, wherein Py is C or T and wherein N is any deoxyribonucleotide.

24. The method of claim 23 wherein said tissue is selected from bone tissue, neuronal tissue, or hepatic tissue.

25. A medicament for repairing bone, neural, or liver damage comprising:
a non-antisense oligonucleotide having about 14 to 40 nucleotides and having at least one subsequence of formula PyNTTTTNT, wherein Py is C or T and wherein N is any deoxyribonucleotide.

26. The medicament of claim 25 wherein said oligonucleotide consists of 14 to 26 nucleotides.

27. The medicament of claim 25 wherein said oligonucleotide has any kind of modification of the natural (phosphodiester) phosphate backbone.

28. The medicament of claim 25 wherein said oligonucleotide has a phosphate backbone modification on the 5′ inter-nucleotide linkages.

29. The medicament of claim 25 wherein said oligonucleotide has a phosphate backbone modification on the 3′ inter-nucleotide linkages.

30. The medicament of claim 25 wherein said oligonucleotide has at least one of the internucleotide linkages as a phosphorotioate linkage.

31. The medicament of claim 25 wherein said oligonucleotide is selected from the group consisting of:

| | |
|---|---|
| TCATCATTTTGTCATTTTGTCATT; | (SEQ ID N^{o} 2) |
| TCCTCCTTTTGTCCTTTTGTCCTT; | (SEQ ID N^{o} 3) |
| TCTTCTTTTTGTCTTTTTGTCTTT; | (SEQ ID N^{o} 4) |
| TTGTTGTTTTGTTGTTTTGTTGTT; | (SEQ ID N^{o} 7) |
| TCATTTTTTTGTTTTTTTGTCATT; | (SEQ ID N^{o} 10) |
| TCATTGTTTTGTTGTTTTGTCATT; | (SEQ ID N^{o} 11) |
| TCATTCTTTTGTTCTTTTGTCATT; | (SEQ ID N^{o} 12) |
| TCATTATTTTGTTATTTTGTCATT; | (SEQ ID N^{o} 15) |
| TCATCCTTTTGTCCTTTTGTCATT; | (SEQ ID N^{o} 17) |
| TCATCTTTTTGTCTTTTTGTCATT; | (SEQ ID N^{o} 18) |
| CATTTTGTTTTTTTTTTTTTTTTT; | (SEQ ID N^{o} 20) |
| TTCATTTTGTTTTTTTTTTTTTTT; | (SEQ ID N^{o} 21) |
| TTTTCATTTTGTTTTTTTTTTTTT; | (SEQ ID N^{o} 22) |
| TTTTTTCATTTTGTTTTTTTTTTT; | (SEQ ID N^{o} 23) |
| TTTTTTTTCATTTTGTTTTTTTTT; | (SEQ ID N^{o} 24) |
| TTTTTTTTTTCATTTTGTTTTTTT; | (SEQ ID N^{o} 25) |
| TTTTTTTTTTTTCATTTTGTTTTT; | (SEQ ID N^{o} 26) |
| TTTTTTTTTTTTTTCATTTTGTTT; | (SEQ ID N^{o} 27) |
| TTTTTTTTTTTTTTTTCATTTTGT; | (SEQ ID N^{o} 28) |
| TTTTCATTTTGTCATTTTGTTTTT; | (SEQ ID N^{o} 29) |
| TCATCAATTTGTCAATTTGTCATT; | (SEQ ID N^{o} 30) |
| ACATCATTTTGTCATTTTGTCATT; | (SEQ ID N^{o} 46) |
| CCATCATTTTGTCATTTTGTCATT; | (SEQ ID N^{o} 47) |
| GCATCATTTTGTCATTTTGTCATT; | (SEQ ID N^{o} 48) |
| TAATCATTTTGTCATTTTGTCATT; | (SEQ ID N^{o} 49) |
| TTATCATTTTGTCATTTTGTCATT; | (SEQ ID N^{o} 50) |
| TGATCATTTTGTCATTTTGTCATT; | (SEQ ID N^{o} 51) |
| TCCTCATTTTGTCATTTTGTCATT; | (SEQ ID N^{o} 52) |
| TCTTCATTTTGTCATTTTGTCATT; | (SEQ ID N^{o} 53) |
| TCAACATTTTGTCATTTTGTCATT; | (SEQ ID N^{o} 55) |
| TCACCATTTTGTCATTTTGTCATT; | (SEQ ID N^{o} 56) |
| TCAGCATTTTGTCATTTTGTCATT; | (SEQ ID N^{o} 57) |
| TCATCATTTTGTCATTTTGTAATT; | (SEQ ID N^{o} 58) |
| TCATCATTTTGTCATTTTGTTATT; | (SEQ ID N^{o} 59) |
| TCATCATTTTGTCATTTTGTGATT; | (SEQ ID N^{o} 60) |
| TCATCATTTTGTCATTTTGTCCTT; | (SEQ ID N^{o} 61) |
| TCATCATTTTGTCATTTTGTCTTT; | (SEQ ID N^{o} 62) |
| TCATCATTTTGTCATTTTGTCAAT; | (SEQ ID N^{o} 64) |
| TCATCATTTTGTCATTTTGTCACT; | (SEQ ID N^{o} 65) |
| TCATCATTTTGTCATTTTGTCAGT; | (SEQ ID N^{o} 66) |
| TCATCATTTTGTCATTTTGTCATA; | (SEQ ID N^{o} 67) |
| TCATCATTTTGTCATTTTGTCATC; | (SEQ ID N^{o} 68) |
| TCATCATTTTGTCATTTTGTCATG; | (SEQ ID N^{o} 69) |
| TCATCAATTGGTCAATTGGTCATT; | (SEQ ID N^{o} 75) |
| TCATCAACTGGTCAACTGGTCATT; | (SEQ ID N^{o} 77) |
| TCATCATTGTGTCATTGTGTCATT; | (SEQ ID N^{o} 84) |
| TCATCATTTGGTCATTTGGTCATT; | (SEQ ID N^{o} 87) |
| TGCTGCTTTTGTGCTTTTGTGCTT; | (SEQ ID N^{o} 91) |
| TCATCATCTTGTCATCTTGTCATT; | (SEQ ID N^{o} 95) |
| TCATCATGTTGTCATGTTGTCATT; | (SEQ ID N^{o} 96) |
| GGGGGTCTTTTTTTCTTTTTTTTT; | (SEQ ID N^{o} 107) |
| TTTTTTCTTTTTTTCTTTTTTGGG; | (SEQ ID N^{o} 108) |
| AAAAATCTTTTTTTCTTTTTTTTT; | (SEQ ID N^{o} 109) |
| TGCTGCTTTTATGCTTTTATGCTT; | (SEQ ID N^{o} 110) |
| TCATCATTCTGTCATTCTGTCATT; | (SEQ ID N^{o} 111) |
| TTTTTTCTTTTTTTCTTTTTTTTT; | (SEQ ID N^{o} 112) |
| TGCTGCTTTTCTGCTTTTCTGCTT; | (SEQ ID N^{o} 113) |
| TTTTTTCTTTTCTTTTTTTTTTTT; | (SEQ ID N^{o} 114) |
| TTTTTCCTTTTTTCCTTTTTTTTT; | (SEQ ID N^{o} 115) |
| CCCCCTCTTTTTTTCTTTTTTTTT; | (SEQ ID N^{o} 116) |
| TTTTTTCTTTTTCTTTTTTTTTTT; | (SEQ ID N^{o} 117) |
| TTTTTTCTTTTTTTCTTTTTTCCC; | (SEQ ID N^{o} 118) |
| TTTTTTCTTTTTCTCTTTTTCTCT; | (SEQ ID N^{o} 119) |
| TTTTTGCTTTTTTGCTTTTTTTTT; | (SEQ ID N^{o} 120) |
| TTTTTACTTTTTTACTTTTTTTTT; | (SEQ ID N^{o} 121) |
| TCATAATTTTGTAATTTTGTCATT; | (SEQ ID N^{o} 122) |
| TTTTTTCTTTTTTTCTTTTTTAAA; | (SEQ ID N^{o} 123) |
| TTTTTTCTTTTTTCTTTTTTTTTT; | (SEQ ID N^{o} 124) |
| TTTTTTTTTTTTCATTTTGTGGGG; | (SEQ ID N^{o} 125) |
| TTTTTTTTTTTTCATTTTGTTTTG; | (SEQ ID N^{o} 126) |
| GGGTTTTTTTTTCATTTTGTTTTT; | (SEQ ID N^{o} 127) |
| GTTTTTTTTTTTCATTTTGTTTTG; | (SEQ ID N^{o} 128) |
| TTTTTTTTTTTTCATTTTGTTTGG; | (SEQ ID N^{o} 129) |
| TTTTTTTTTTTTCATTTTGTTTTA; | (SEQ ID N^{o} 130) |
| TTTTTTTTTTTTCATTTTGTTTGA; | (SEQ ID N^{o} 131) |
| TTTTTTTTTTTTCATTTTGTTTAG; | (SEQ ID N^{o} 132) |
| TTTTTTTTTTTTCATTTTGTTTAA. | (SEQ ID N^{o} 133) |

32. The method of claim 23 wherein said patient is a human.

33. The medicament of claim 25 wherein said oligonucleotide is included in a pharmaceutically acceptable carrier.

34. The medicament of claim 25 wherein said oligonucleotide is encapsulated in a slow release delivery vehicle.

35. The method of claim 23 wherein said oligonucleotide is administered to the patient in combination with a device aimed to aid in the treatment of a tissue or organ deficiency or alteration.

36. The medicament of claim 25 wherein said oligonucleotide is present in amounts of 1 μg to 100 mg per dose.

37. The medicament of claim 25 wherein said medicament is selected from the group consisting of liquid, gel and lyophilized formulations.

38. The medicament of claim 25 said medicament is administered by intradermic, intramuscular or intravenous injection.

39. The medicament of claim 25 wherein said medicament is administered by an oral, intranasal, anal, vaginal, or trans-dermal route.

40. The medicament of claim 25 said oligonucleotide consists of 25 or less nucleotides.


SEQUENCE LISTING (133 sequences)

SEQ ID NO: 1 - DNA, 24 bp, Artificial
  Synthetic Construct
tgctgcttttgtgcttttgtgctt

SEQ ID NO: 2 - DNA, 24 bp, Artificial
  Synthetic Construct
tcatcattttgtcattttgtcatt

SEQ ID NO: 3 - DNA, 24 bp, Artificial
  Synthetic Construct
tcctccttttgtccttttgtcctt

SEQ ID NO: 4 - DNA, 24 bp, Artificial
  Synthetic Construct
tcttctttttgtctttttgtcttt

SEQ ID NO: 5 - DNA, 24 bp, Artificial
  Synthetic Construct
tagtagttttgtagttttgtagtt

SEQ ID NO: 6 - DNA, 24 bp, Artificial
  Synthetic Construct
tggtggttttgtggttttgtggtt

SEQ ID NO: 7 - DNA, 24 bp, Artificial
  Synthetic Construct
ttgttgttttgttgttttgttgtt

SEQ ID NO: 8 - DNA, 24 bp, Artificial
  Synthetic Construct
tgctgcaaaagagcaaaagagcaa

SEQ ID NO: 9 - DNA, 24 bp, Artificial
  Synthetic Construct
tgctgcccccgcgcccccgcgccc

SEQ ID NO: 10 - DNA, 24 bp, Artificial
  Synthetic Construct
tcatttttttgtttttttgtcatt

SEQ ID NO: 11 - DNA, 24 bp, Artificial
  Synthetic Construct
tcattgttttgttgttttgtcatt

SEQ ID NO: 12 - DNA, 24 bp, Artificial
  Synthetic Construct
tcattcttttgttcttttgtcatt

SEQ ID NO: 13 - DNA, 24 bp, Artificial
  Synthetic Construct
aaaaaactaaaaaaaactaaaaaa

SEQ ID NO: 14 - DNA, 24 bp, Artificial
  Synthetic Construct
tcataattttgtaattttgtcatt

SEQ ID NO: 15 - DNA, 24 bp, Artificial
  Synthetic Construct
tcattattttgttattttgtcatt

SEQ ID NO: 16 - DNA, 24 bp, Artificial
  Synthetic Construct
tcatgattttgtgattttgtcatt

SEQ ID NO: 17 - DNA, 24 bp, Artificial
  Synthetic Construct
tcatccttttgtccttttgtcatt

SEQ ID NO: 18 - DNA, 24 bp, Artificial
  Synthetic Construct
tcatctttttgtctttttgtcatt

SEQ ID NO: 19 - DNA, 24 bp, Artificial
  Synthetic Construct
tttttttttttttttttttttttt

SEQ ID NO: 20 - DNA, 24 bp, Artificial
  Synthetic Construct
cattttgttttttttttttttttt

SEQ ID NO: 21 - DNA, 24 bp, Artificial
  Synthetic Construct
ttcattttgttttttttttttttt

SEQ ID NO: 22 - DNA, 24 bp, Artificial
  Synthetic Construct
ttttcattttgttttttttttttt

SEQ ID NO: 23 - DNA, 24 bp, Artificial
  Synthetic Construct
ttttttcattttgttttttttttt

SEQ ID NO: 24 - DNA, 24 bp, Artificial
  Synthetic Construct
ttttttttcattttgttttttttt

SEQ ID NO: 25 - DNA, 24 bp, Artificial
  Synthetic Construct
ttttttttttcattttgttttttt

SEQ ID NO: 26 - DNA, 24 bp, Artificial
  Synthetic Construct
ttttttttttttcattttgttttt

SEQ ID NO: 27 - DNA, 24 bp, Artificial
  Synthetic Construct
ttttttttttttttcattttgttt

SEQ ID NO: 28 - DNA, 24 bp, Artificial
  Synthetic Construct
ttttttttttttttttcattttgt

SEQ ID NO: 29 - DNA, 24 bp, Artificial
  Synthetic Construct
ttttcattttgtcattttgttttt

SEQ ID NO: 30 - DNA, 24 bp, Artificial
  Synthetic Construct
tcatcaatttgtcaatttgtcatt

SEQ ID NO: 31 - DNA, 24 bp, Artificial
  Synthetic Construct
tcatcatattgtcatattgtcatt

SEQ ID NO: 32 - DNA, 24 bp, Artificial
  Synthetic Construct
tcatcattatgtcattatgtcatt

SEQ ID NO: 33 - DNA, 24 bp, Artificial
  Synthetic Construct
tcatcatttagtcatttagtcatt

SEQ ID NO: 34 - DNA, 24 bp, Artificial
  Synthetic Construct
tcatcattttatcattttatcatt

SEQ ID NO: 35 - DNA, 24 bp, Artificial
  Synthetic Construct
tcatcattttttcattttttcatt

SEQ ID NO: 36 - DNA, 24 bp, Artificial
  Synthetic Construct
tcatcattttctcattttctcatt

SEQ ID NO: 37 - DNA, 24 bp, Artificial
  Synthetic Construct
tcatcattttgacattttgacatt

SEQ ID NO: 38 - DNA, 24 bp, Artificial
  Synthetic Construct
tcatcatttagacatttagacatt

SEQ ID NO: 39 - DNA, 24 bp, Artificial
  Synthetic Construct
tcatcattatgacattatgacatt

SEQ ID NO: 40 - DNA, 24 bp, Artificial
  Synthetic Construct
tcatcatattgacatattgacatt

SEQ ID NO: 41 - DNA, 24 bp, Artificial
  Synthetic Construct
tcatcattaagacattaagacatt

SEQ ID NO: 42 - DNA, 24 bp, Artificial
  Synthetic Construct
tcatcatatagacatatagacatt

SEQ ID NO: 43 - DNA, 24 bp, Artificial
  Synthetic Construct
tcatcataatgacataatgacatt

SEQ ID NO: 44 - DNA, 24 bp, Artificial
  Synthetic Construct
tcatcataaagacataaagacatt

SEQ ID NO: 45 - DNA, 24 bp, Artificial
  Synthetic Construct
tcatcaaaaagacaaaaagacatt

SEQ ID NO: 46 - DNA, 24 bp, Artificial
  Synthetic Construct
acatcattttgtcattttgtcatt

SEQ ID NO: 47 - DNA, 24 bp, Artificial
  Synthetic Construct
ccatcattttgtcattttgtcatt

SEQ ID NO: 48 - DNA, 24 bp, Artificial
  Synthetic Construct
gcatcattttgtcattttgtcatt

SEQ ID NO: 49 - DNA, 24 bp, Artificial
  Synthetic Construct
taatcattttgtcattttgtcatt

SEQ ID NO: 50 - DNA, 24 bp, Artificial
  Synthetic Construct
ttatcattttgtcattttgtcatt

SEQ ID NO: 51 - DNA, 24 bp, Artificial
  Synthetic Construct
tgatcattttgtcattttgtcatt

SEQ ID NO: 52 - DNA, 24 bp, Artificial
  Synthetic Construct
tcctcattttgtcattttgtcatt

SEQ ID NO: 53 - DNA, 24 bp, Artificial
  Synthetic Construct
tcttcattttgtcattttgtcatt

SEQ ID NO: 54 - DNA, 24 bp, Artificial
  Synthetic Construct
tcgtcattttgtcattttgtcatt

SEQ ID NO: 55 - DNA, 24 bp, Artificial
  Synthetic Construct
tcaacattttgtcattttgtcatt

SEQ ID NO: 56 - DNA, 24 bp, Artificial
  Synthetic Construct
tcaccattttgtcattttgtcatt

SEQ ID NO: 57 - DNA, 24 bp, Artificial
  Synthetic Construct
tcagcattttgtcattttgtcatt

SEQ ID NO: 58 - DNA, 24 bp, Artificial
  Synthetic Construct
tcatcattttgtcattttgtaatt

SEQ ID NO: 59 - DNA, 24 bp, Artificial
  Synthetic Construct
tcatcattttgtcattttgttatt

SEQ ID NO: 60 - DNA, 24 bp, Artificial
  Synthetic Construct
tcatcattttgtcattttgtgatt

SEQ ID NO: 61 - DNA, 24 bp, Artificial
  Synthetic Construct
tcatcattttgtcattttgtcctt

SEQ ID NO: 62 - DNA, 24 bp, Artificial
  Synthetic Construct
tcatcattttgtcattttgtcttt

SEQ ID NO: 63 - DNA, 24 bp, Artificial
  Synthetic Construct
tcatcattttgtcattttgtcgtt

SEQ ID NO: 64 - DNA, 24 bp, Artificial
  Synthetic Construct
tcatcattttgtcattttgtcaat

SEQ ID NO: 65 - DNA, 24 bp, Artificial
  Synthetic Construct
tcatcattttgtcattttgtcact

SEQ ID NO: 66 - DNA, 24 bp, Artificial
  Synthetic Construct
tcatcattttgtcattttgtcagt

SEQ ID NO: 67 - DNA, 24 bp, Artificial
  Synthetic Construct
tcatcattttgtcattttgtcata

SEQ ID NO: 68 - DNA, 24 bp, Artificial
  Synthetic Construct
tcatcattttgtcattttgtcatc

SEQ ID NO: 69 - DNA, 24 bp, Artificial
  Synthetic Construct
tcatcattttgtcattttgtcatg

SEQ ID NO: 70 - DNA, 12 bp, Artificial
  Synthetic Construct
ttttcattttgt

SEQ ID NO: 71 - DNA, 16 bp, Artificial
  Synthetic Construct
ttttcattttgttttt

SEQ ID NO: 72 - DNA, 20 bp, Artificial
  Synthetic Construct
ttttcattttgttttttttt

SEQ ID NO: 73 - DNA, 24 bp, Artificial
  Synthetic Construct
ttttttttttttcattttgttttt

SEQ ID NO: 74 - DNA, 28 bp, Artificial
  Synthetic Construct
ttttcattttgttttttttttttttttt

SEQ ID NO: 75 - DNA, 24 bp, Artificial
  Synthetic Construct
tcatcaattggtcaattggtcatt

SEQ ID NO: 76 - DNA, 24 bp, Artificial
  Synthetic Construct
tcatcaaatggtcaaatggtcatt

SEQ ID NO: 77 - DNA, 24 bp, Artificial
  Synthetic Construct
tcatcaactggtcaactggtcatt

SEQ ID NO: 78 - DNA, 24 bp, Artificial
  Synthetic Construct
tcatcaagtggtcaagtggtcatt

SEQ ID NO: 79 - DNA, 24 bp, Artificial
  Synthetic Construct
tcatcaataggtcaataggtcatt

SEQ ID NO: 80 - DNA, 24 bp, Artificial
  Synthetic Construct
tcatcaatcggtcaatcggtcatt

SEQ ID NO: 81 - DNA, 24 bp, Artificial
  Synthetic Construct
tcatcaatgggtcaatgggtcatt

SEQ ID NO: 82 - DNA, 24 bp, Artificial
  Synthetic Construct
tcatcattatgtcattatgtcatt

SEQ ID NO: 83 - DNA, 24 bp, Artificial
  Synthetic Construct
tcatcattctgtcattctgtcatt

SEQ ID NO: 84 - DNA, 24 bp, Artificial
  Synthetic Construct
tcatcattgtgtcattgtgtcatt

SEQ ID NO: 85 - DNA, 24 bp, Artificial
  Synthetic Construct
tcatcatttagtcatttagtcatt

SEQ ID NO: 86 - DNA, 24 bp, Artificial
  Synthetic Construct
tcatcatttcgtcatttcgtcatt

SEQ ID NO: 87 - DNA, 24 bp, Artificial
  Synthetic Construct
tcatcatttggtcatttggtcatt

SEQ ID NO: 88 - DNA, 24 bp, Artificial
  Synthetic Construct
tcatcattttgacattttgacatt

SEQ ID NO: 89 - DNA, 24 bp, Artificial
  Synthetic Construct
tcatcattttgccattttgccatt

SEQ ID NO: 90 - DNA, 24 bp, Artificial
  Synthetic Construct
tcatcattttggcattttggcatt

SEQ ID NO: 91 - DNA, 24 bp, Artificial
  Synthetic Construct
tgctgcttttgtgcttttgtgctt

SEQ ID NO: 92 - DNA, 24 bp, Artificial
  Synthetic Construct
tcatcactttggcactttggcatt

SEQ ID NO: 93 - DNA, 24 bp, Artificial
  Synthetic Construct
tcatcagtttggcagtttggcatt

SEQ ID NO: 94 - DNA, 24 bp, Artificial
  Synthetic Construct
tcatcatattggcatattggcatt

SEQ ID NO: 95 - DNA, 24 bp, Artificial
  Synthetic Construct
tcatcatcttggcatcttggcatt

SEQ ID NO: 96 - DNA, 24 bp, Artificial
  Synthetic Construct
tcatcatgttggcatgttggcatt

SEQ ID NO: 97 - DNA, 24 bp, Artificial
  Synthetic Construct
tgctgcccccgcgcccccgcgccc

SEQ ID NO: 98 - DNA, 24 bp, Artificial
  Synthetic Construct
ggggtttttttttttttttttttt

SEQ ID NO: 99 - DNA, 24 bp, Artificial
  Synthetic Construct
ggggtctttttttctttttttctt

SEQ ID NO: 100 - DNA, 24 bp, Artificial
  Synthetic Construct
tgctgcccccgcgcccccgcgccc

SEQ ID NO: 101 - DNA, 24 bp, Artificial
  Synthetic Construct
ggggacttttatacttttatactt

SEQ ID NO: 102 - DNA, 24 bp, Artificial
  Synthetic Construct
ggggtatttttttatttttttatt

SEQ ID NO: 103 - DNA, 24 bp, Artificial
  Synthetic Construct
gggggcttttgggcttttgggcgg

SEQ ID NO: 104 - DNA, 24 bp, Artificial
  Synthetic Construct
gggggctttagtgctttagtgctt

SEQ ID NO: 105 - DNA, 24 bp, Artificial
  Synthetic Construct
gggggctaatgtgctaatgtgctt

SEQ ID NO: 106 - DNA, 24 bp, Artificial
  Synthetic Construct
tcgtcgaaaagtcgaaaagtcgaa

SEQ ID NO: 107 - DNA, 24 bp, Artificial
  Synthetic Construct
gggggtctttttttcttttttttt

SEQ ID NO: 108 - DNA, 24 bp, Artificial
  Synthetic Construct
ttttttctttttttcttttttggg

SEQ ID NO: 109 - DNA, 24 bp, Artificial
  Synthetic Construct
aaaaatctttttttcttttttttt

SEQ ID NO: 110 - DNA, 24 bp, Artificial
  Synthetic Construct
tgctgcttttatgcttttatgctt

SEQ ID NO: 111 - DNA, 24 bp, Artificial
  Synthetic Construct
tcatcattctgtcattctgtcatt

SEQ ID NO: 112 - DNA, 24 bp, Artificial
  Synthetic Construct
ttttttctttttttcttttttttt

SEQ ID NO: 113 - DNA, 24 bp, Artificial
  Synthetic Construct
tgctgcttttctgcttttctgctt

SEQ ID NO: 114 - DNA, 24 bp, Artificial
  Synthetic Construct
ttttttcttttctttttttttttt

SEQ ID NO: 115 - DNA, 24 bp, Artificial
  Synthetic Construct
tttttccttttttccttttttttt

SEQ ID NO: 116 - DNA, 24 bp, Artificial
  Synthetic Construct
ccccctctttttttcttttttttt

SEQ ID NO: 117 - DNA, 24 bp, Artificial
  Synthetic Construct
ttttttctttttcttttttttttt

SEQ ID NO: 118 - DNA, 24 bp, Artificial
  Synthetic Construct
ttttttctttttttcttttttccc

SEQ ID NO: 119 - DNA, 24 bp, Artificial
  Synthetic Construct
ttttttctttttctctttttctct

SEQ ID NO: 120 - DNA, 24 bp, Artificial
  Synthetic Construct
tttttgcttttttgcttttttttt

SEQ ID NO: 121 - DNA, 24 bp, Artificial
  Synthetic Construct
tttttacttttttacttttttttt

SEQ ID NO: 122 - DNA, 23 bp, Artificial
  Synthetic Construct
cataattttgtaattttgtcatt

SEQ ID NO: 123 - DNA, 24 bp, Artificial
  Synthetic Construct
ttttttctttttttcttttttaaa

SEQ ID NO: 124 - DNA, 24 bp, Artificial
  Synthetic Construct
ttttttcttttttctttttttttt

SEQ ID NO: 125 - DNA, 24 bp, Artificial
  Synthetic Construct
ttttttttttttcattttgtgggg

SEQ ID NO: 126 - DNA, 24 bp, Artificial
  Synthetic Construct
ttttttttttttcattttgttttg

SEQ ID NO: 127 - DNA, 24 bp, Artificial
  Synthetic Construct
gggtttttttttcattttgttttt

SEQ ID NO: 128 - DNA, 24 bp, Artificial
  Synthetic Construct
gtttttttttttcattttgttttg

SEQ ID NO: 129 - DNA, 24 bp, Artificial
  Synthetic Construct
ttttttttttttcattttgtttgg

SEQ ID NO: 130 - DNA, 24 bp, Artificial
  Synthetic Construct
ttttttttttttcattttgtttta

SEQ ID NO: 131 - DNA, 24 bp, Artificial
  Synthetic Construct
ttttttttttttcattttgtttga

SEQ ID NO: 132 - DNA, 24 bp, Artificial
  Synthetic Construct
ttttttttttttcattttgtttag

SEQ ID NO: 133 - DNA, 24 bp, Artificial
  Synthetic Construct
ttttttttttttcattttgtttaa